(19) United States
(12) Patent Application Publication
(10) Pub. No.: US 2002/0054888 A1
(43) Pub. Date: May 9, 2002

(54) Dissociated pili, their production and use

(76) Inventors: Peter O'Hanley, Washington, DC (US); Kenneth Denich, Edmonton (CA)

Correspondence Address: Stephen B. Maebius, FOLEY & LARDNER, Suite 500, 3000 K Street, N.W., Washington, DC, 20007-5109, US

(21) Appl. No.: 09/833,067

(22) Filed: Apr. 12, 2001

Related U.S. Application Data
(60) Provisional application No. 60/196,493, filed on Apr. 12, 2000.

(51) Int. Cl. A61K 39/02; C12N 1/20

(52) U.S. Cl. 424/242.1; 435/252.1

(57) ABSTRACT

A method of producing pili and vaccines containing pili is described using bacteria harboring mutations that facilitate detachment of pili from the bacteria. Wild type pili have known immunoprotective effects in treating urinary tract infections. The mutant pili produced by this method are also shown to have such immunoprotective effects. Therefore, the pili may be used to make vaccines for treating urinary tract infections.

### FIELD OF THE INVENTION

The present invention relates to pili-based vaccines, their production, and use, and more particularly, to the production of pili through the use of pilus-producing strains having at least one mutation that leads to a greater number of pili dissociated from the cell surface compared to a wild type.

### BACKGROUND OF THE INVENTION

U.S. Pat. No. 4,740,585 discloses peptide vaccines for urinary tract infections prepared from synthetic peptides based on short sequences contained in HUR849 pilin A.

U.S. Pat. No. 4,736,017 discloses peptide vaccines for urinary tract infections prepared from purified whole Gal-Gal pilus proteins or fragments thereof.

Baga et al., Cell, 49(1987): 241-251, disclose papH deletion mutants in one strain of Escherichia coli. The reference indicates that, in these papH deletion mutants, 50%-70% of total pilus antigen was found free of cells in the form of polymerized structures. Further, the dissociated and purified pili from these mutants are stated to agglutinate erythrocytes, though data is not disclosed. The reference does not indicate whether such dissociated pili from the from the papH mutants in polymerized form are effective as vaccines or whether these mutant pili would have altered antigenicity. In addition, the reference does not give the exact sequences of the two deletion mutants from the one strain of E. coli.

### SUMMARY OF THE INVENTION

An embodiment of the present invention is an immunogenic composition, including a vaccine for E. coli urinary tract infections, comprising dissociated pili from a pilus-producing bacteria having at least one mutation that facilitates detachment of the pili from the cell surface of the bacteria relative to a wild type strain.

Another embodiment of the present invention is a process for producing pili and vaccines comprising pili (including vaccines for urinary tract infections) comprising culturing a pilus-producing bacteria having at least one mutation that facilitates detachment of the pili from the cell surface of the bacteria relative to a wild type strain, recovering dissociated pili, and formulating a vaccine comprising the pili.

Another embodiment of the present invention is a method of treating or preventing a urinary tract infection comprising administering to a subject in need thereof a vaccine produced according to the invention.

Another embodiment of the present invention are E. coli bacteria having novel mutations that facilitate detachment of pili from the cell surface of the bacteria relative to a wild type strain.

### BRIEF DESCRIPTION OF THE DRAWINGS

FIG. 1 shows a genetic and physical map of recombinant plasmids used in the present invention.

FIG. 2(a) provides the DNA sequence of pHUR849 papH, FIG. 2(b) provides the DNA sequence of pDAL201B papH, FIG. 2(c) provides the DNA sequence of pDAL210B papH, and FIG. 2(d) provides the DNA sequence of pDAL200A papH.

FIG. 3 provides a comparison of the papH DNA sequences of pHUR849, pDAL200A, pDAL201B, and pDAL210B.

FIG. 4 gives a comparison of deduced amino acid sequences of papH genes pHUR849, pDAL200A, pDAL201B, and pDAL210B.

FIG. 5(a) shows the amino acids (which are underlined) that are deleted from papH in pHUR849 and FIG. 5(b) shows the amino acids (which are underlined) that are deleted from papH in pDAL201B, pDAL210B, and pDAL200A.

### DETAILED DESCRIPTION OF THE PREFERRED EMBODIMENT

An embodiment of the present invention is an immunogenic composition, including a vaccine for urinary tract infections, comprising dissociated pili from a pilus-producing bacteria having at least one mutation that facilitates detachment of the pili from the surface of bacteria relative to a wild type strain. Preferably, the mutation is one that impairs or eliminates the anchoring function of papH.

Another embodiment of the present invention is a process for producing pili or a vaccine comprising pili for urinary tract infections comprising culturing a pilus-producing bacteria having at least one mutation that facilitates detachment of the pili from the bacteria and recovering detached pili from the culture, preferably from the culture supernatant. Optionally, the method further comprises formulating a vaccine comprising the pili. Preferably, detached pili are recovered from the culture supernatant by centrifugation and subsequently purified by cycles of magnesium sulfate precipitation and tris solubilization according to known techniques for purifying or isolating pili.

In one particular embodiment of the pili production method, a one-liter TSB culture yields about 10 mg of purified pili after 18 hours from each of the 4 recombinant strains that harbor papH mutations.

Another embodiment of the present invention is a method of treating or preventing a urinary tract infection comprising administration to a subject in need thereof a vaccine produced according to the invention.

Another embodiment of the present invention are E. coli bacteria having novel mutations that facilitate detachment of pili from the surface of bacteria by impairing or eliminating the anchoring function of PapH.

Preferably, the pilus-producing bacteria of the invention is an E. coli strain, more preferably those disclosed in the examples below.

In the present invention, any mutation may be used that facilitates detachment of pili from the bacteria relative to a wild type strain. Preferably, the mutation impairs the anchoring function of PapH relative to a wild type pilus-producing bacteria, thereby increasing the amount of dissociated pili from the bacteria found in the culture supernatant relative to a wild type pilus-producing bacteria. Preferably, the mutation is a deletion mutation in the DNA encoding PapH, but other types of mutations achieving the same function may be used, such as insertion mutations.

Vaccines comprising the dissociated pili of the invention are formulated according to known methods, including those described in U.S. Pat. No. 4,736,017. Suitable adjuvants may be used in the vaccines. The method of preventing E. coli urinary tract infections includes those described in U.S. Pat. No. 4,736,017.

Adherence of Eschefichia coli to uroepithelial cells is an important pathogenic step in the development of urinary tract infections. There are a number of adhesins expressed by uropathogenic E. coli, which may mediate uroepithelial attachment; however, pyelonephritogenic strains are characterized by the high frequency of pili associated with the α-D-Galp-(1-4)-β-D-Galp (Gal-Gal) binding. The Gal-Gal binding phenotype is considered critical to the pathogenesis of unobstructive, ascending urinary tract infection in anatomically normal, otherwise healthy young women. This is probably due to the absence of a natural host defense factor in urine to prevent the Gal-Gal binding to uroepithelial cells. Digalactoside-binding adherence is mediated by pili, which are also known as Pap pili, or P pili because they bind to the P₁ blood group antigen (a globoside containing Gal-Gal) that is present on human erythrocytes and all epithelial cells.

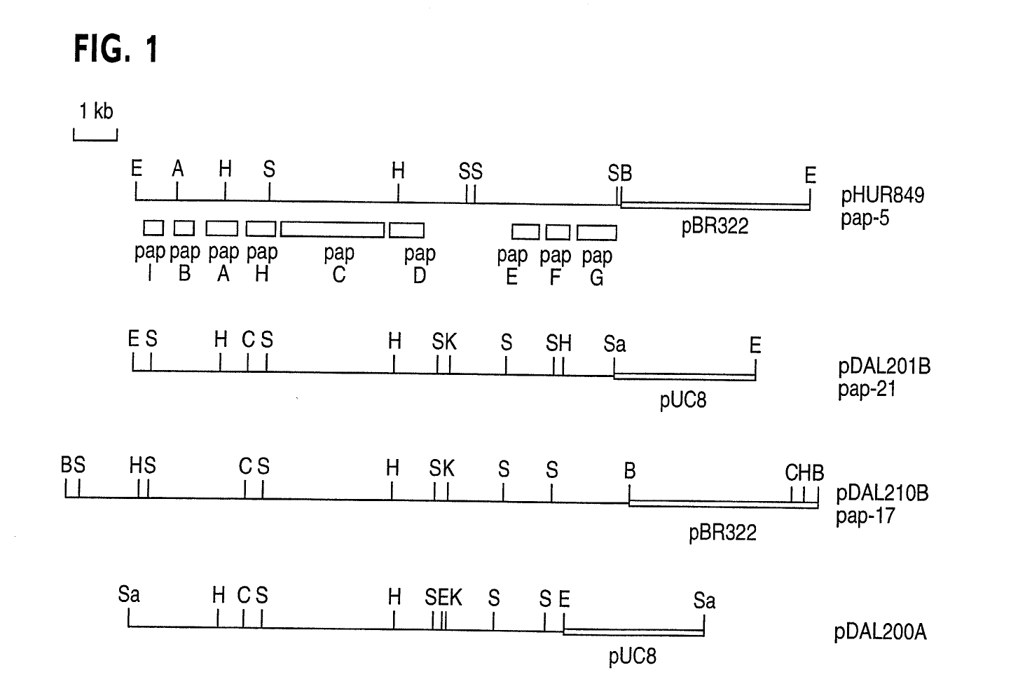

The pap operon consists of at least 9 genes (1) that are required for the expression of the Pap pilus-adhesion complex (see FIG. 1). PapA is the major (structural) fimbrial subunit. PapH is involved in both the termination of pilus growth and is required to anchor the fully grown pilus to the cell surface. PapC is located in the outer membrane and forms the assembly platform for pilus growth. PapD is a periplasmic protein that forms complexes intracellularly with the pilus subunits before assembly. PapE, PapF, and PapG are tip pilus components. PapG is the adhesion molecule conferring Gal-Gal binding specificity. PapF complexes with PapG, and PapE attaches to PapA moieties, as well as, attaches and orients the PapF-PapG complex so that the adhesin is at the tip of the pilus.

The present invention is further illustrated by, though in no way limited to, the following examples.

In the following examples, mutagenesis of the papH structural gene, which is responsible for anchoring the globoside-binding pili to the cell surface, is utilized. The papH gene was mutagenized in 4 Gal-Gal pilus-binding plasmids, [pHUR849 (pap-5), pDAL201B (Pap-21), pDAL210B (pap-17), and pDAL200A (pap-200A)], which encode for the serotypes F13, F7₁, F7₂, and F9 (2) in transformants, respectively. This was accomplished by creating deletions of 237 or 300-bp within the papH gene of each strain. These deletions encode for 79 or 100 amino acids respectively, and lead to a truncated form of the PapH protein that allows for the mutant recombinant piliated strains to secrete newly synthesized pili into the culture medium. Since PapH or its truncated form is not required in the liberation or the assembly of the growing pilin subunits (3), the growing Pap pilus can be detached because of unstable interaction between PapA and the cell envelope. In addition, the complete nucleotide and deduced amino acid sequences of papH genes in all 4 recombinant strains and their deletion derivatives are disclosed herein.

### Experimental Procedures

Bacterial strains, plasmids, and growth conditions

Bacterial strains and plasmids used for these examples are listed in Table 1 (below) and FIG. 1. The source of the chromosal DNA for pDAL210B was E. coli strain 3669, originally isolated from a woman with acute pyeionephritis (2). The source of the chromosomal DNA for pDAL210B and pDAL210B was E. coli strain C1212, originally isolated from a woman with acute cystitis (2). The source of the chromosomal DNA for pHUR849 was isolated from E. coli strain J96, originally isolated from a woman with acute pyelonephritis (4). All bacteria were cultured in Luria broth or on Luria-agar plates, containing 40 μg/ml X-gal and 20 mM IPTG. Antibiotics were used at the indicated final concentrations: ampicillin, 100 μg/ml, and tetracycline 34 μg/ml, for the selection of plasmid-containing strains. Bacterial transformation were performed as previously described (5).

TABLE 1
Plasmids used In this study

| Plasmid | Description | Reference or source |
|---|---|---|
| E. coli | | |
| HB101 | F^{-Δ(gpt-pro)62, leu B6, sup E44, ara- -4, galK2. lac Y1} | Stratagene |
| | Δ(mcrC-mmr), rsp L20(Str^{r}), xyl-5. mti-1. recA13 | |
| XL-1 Blue | recA⁻( recA1, lac. end A1, gyr A96, thi 1, hsd r R7, | Stratagene |
| | supE44, relAl. (P proAB, iaciq, laoZ.ΔM15, Tn10)) | |
| SURE | e14⁻(mcr A), Δ(mcrCB-hsd SMR-mmr)171, end Al. sup | Stratagene |
| | E44, th1. gyrA96, rel Al, lac. wcfl, recJ, sbcC, umuC | |
| | :;Tn5(kan^{r}), uvr C, [F proAB. lac iq, lac ZΔM15, Tn10] | |
| DL844 | DL1784 irp:mTn10 | D. Low |
| Plasmids | | |
| pDAL200A | pUC8 containing a 9 kb Sau 3a DNA fragment encoding | (2) |
| | for the pap-200A operon DNA sequence (see FIG. 1) | |
| pDAL210B | pUC8 containing all kb Eco RI-Sal I DNA tragment | (2) |
| | encoding for the pap-21 operon DNA sequence | |
| | (see FIG. 1) | |
| pDAL210B | pBR322 containing a 13.5 kb Bam HI DNA fragment | (2) |
| | encoding for the pap-17 operon DNA sequence | |
| | (aee FIG. 1) | |
| pHUR849 | pBR322 containing a 11.1 kb Eco RI-Bam HI DNA | (4) |
| | fragment encoding for the pap-5 operon DNA sequence | |
| | (see FIG. 1) | |
| pKTD-1 | pBluescript II containing a 16-bp deletion which removes | This study |
| | the Xba I-Sma I multiple cloning site (MCS),of the vector | |
| | SK^{-a} | |
| pKTD-2 | pKTD-1 containing a 8-bp deletion which removes the Cla | This study |
| | I-Hinc II MCS 01 the vector 8K- | |
| pKTD-3 | pBluescript II containing a 8-bp deletion Which removes | This study |
| | the Eco Ri-Hind III MCS of the vector SK- | |
| pKD201B-1 | pBluescript II containing a 7.1 kb Eco RI.Kpn I pap-17 DNA | This study |
| | fragment derived from pDAL210B | |
| pKO201B-2 | pKTD-2 containing a 4.1 kb Hind III DNA fragment derived | This study |
| | from pKD201B-t | |
| pKD201B4 | pkD2018-2 containing 3.86kb Hind III DNA fragment, | This study |
| | which contains a 237-bp CIa I-Sins I deletion of papH, | |
| | derived from pKD2018-2 | |
| pKD201B-4 | pBluescript II containing a 237bp Cla I-Sm. I DNA | This study |
| | fragment derived from pKD2015-2 | |
| pKD201B-5 | pBlusscript II containIng 5700 bp Eco RI-Cla I DNA | This study |
| | fragment derived from pKD2OI9-2 | |
| pKD201B-6 | pKD201B-2 containing a 3.2 kb DNA fragment, which | This study |
| | contains a 937-bp Eco RI-Sins I deletion | |
| pKD201B-7 | pBluescript II containing a 8.86kb Eco RI-Kpn I DNA | This study |
| | fragment, which resulted from the ligation of a 3.86 kb | |
| | Hind Ill DNA fragment derived from pKD20154, which | |
| | contains a 237-bp deletion of papH. to a 3 kd DNA | |
| | fragment, derived from pKD201B-I | |
| pKD201B-8 | pUC8 containing a 10.86kb pap21 operon DNA | This study |
| | sequence, which resulted from tne ligation of a 8.86 Kb | |
| | Eco RI-Kpn I fragment derived from pKD201B-7. which | |
| | contains a 237-bp deletion of pap H. to a 4 Kb EGo RI-Sal I | |
| | fragment, derived horn and pDAL210B | |
| pKD200A-1 | pKTD-3 containing a 6.4 Kb Sal I- Kpn I fragment derived | This study |
| | from pDAL200A | |
| pKD200A-2 | pKTD-2 containing a 4.1 Kb Hind III fragment derived from | This study |
| | pKD200A-1 | |
| pKD200A-3 | pKD200A-2 contaIning 3.86 Kb Hind ill DNA fragment. | This study |
| | which contains a 237-bp Cia 1-Sma I deletion of pap H | |
| PKD200A-4 | pBluescript II containing a 237-bp CIa -Sma I DNA | This study |
| | fragment derived from pKD200A-2 | |
| pKD200A-5 | pBluescript H containing a 700.bp Eco Ri-Cia I DNA | This study |
| | fragment derived from pKD200A-2 | |
| pKD200A-6 | pKD200A-2 containing a 3.18 Kb DNA fragment, which | This study |
| | contains a 937-bp EGO RI-Sma I deletion | |
| pKD200A-7 | pBluescript II containing a 6.18Kb Sal I-Kpn I DNA | This study |
| | fragment, which resulted from the ligation of a 3.86 Kb | |
| | Hind III DNA fragment derived from pKD200A-3 to a 2.3 kd | |
| | Sal I-Kpn I DNA fragment, derived from pKD200A-1 | |
| pKD200A-8 | pUC8 containing a 8.76 Kb pap 200A operon DNA | This study |
| | sequence, which resulted from the ligation of a 2.8 Kb Sat | |
| | I-Kpn I DNA fragment derived pDAL200A, to 6.16kb Sal | |
| | I-Kpn I DNA fragment, which contains a 237-bp deletion of | |
| | papH | |
| pKD210B-1 | pKTD-3 containing 13.5 kb Bam HI DNA fragment | This study |
| | encoding for the pap-17 operon DNA sequence derived | |
| | from pDAL210B | |
| pKD210B-2 | pKTD-2 containing a 6 kb Hind II DNA fragment derived | This study |
| | from pKD2108-1 | |
| pKD210B-3 | pBluescript II containing a 237bp Cla 1-Sma 1 DNA | This study |
| | fragment derived from pKD210B-2 | |
| pKD210B-4 | pBluescript II containing a 2.5 kb Eco RI-Cla I DNA | This study |
| | fragment derived from pKD210B-2 | |
| pKD210B-5 | pBluescript II containing a 3.26 kb CIa I-Kpn I DNA | This study |
| | fragment derived from pKD210B-2 | |
| pKD210B-6 | pBluescript II containing a 2.5 kb Eco RI-Sma I fusion-Hind | This study |
| | III DNA fragment derived from pKD210B-5 | |
| pKD210B-7 | pBluescript II containing a 2.5 kb Bam HI-Kind III DNA | This study |
| | fragment derived from pKD210B-1 | |
| pKD210B-8 | pBluescript II containing a 3.3 kb Sma I-Kpn I DNA | This study |
| | fragment derived from pKD210B-5 | |
| pKD210B-9 | pBluescript II containing a 5.77 kb Eco Ri-Kpn I DNA | This study |
| | fragment resulting from the ilgation of a 3.26 kb Sma I-KPN | |
| | I DNA fragment derived from pKD210B-5, to pKD210B-4 | |
| | linearized with Cla I-Kpn I, the fusion of Cla I-Sma I creates | |
| | a 237-bp deletion of papH | |
| pKD210B-10 | pBluescript II containing a 13.3 kb Bam HI DNA fragment | This study |
| | derived from the ligation of a 5.76 kb Hind III DNA fragment | |
| | derived from pKD210B-9, to a 7.5 hb Hind III DNA | |
| | fragment derived from pKD0210B-1 | |
| pKD210B-11 | pBRS22 containing a 13.3 kb Bam HI DNA fragment pap | This study |
| | -17 operon DNA sequence derived from pKD210B-10 | |
| | which contains a 237 bp deletion of papH | |
| pKD849-1 | pKTD-2 containing a 4.1 kb Hind III DNA fragment denved | This study |
| | from pHUR 849 | |
| pKD849-2 | pBluescript II containing a 4.1 kb Hind III DNA fragment | This study |
| | derived from pHUR 849 | |
| pKD849-3 | pBluescript II containing a 3.2 kb Sma I DNA fragment | This study |
| | derived from pKD849-2 | |
| pKD849-4 | pBluescript II containing a 3.85 kb DNA fragment derived | This study |
| | from the ligation of a 645-bp PCR product derived from | |
| | pHUR 849, to pKD849-3 linearized with Pma I, which | |
| | contains a 300-bp deletion of papH | |
| pKD849-5 | pBR322 containing a 10.8 kb Eco RI-Bam HI DNA | This study |
| | fragment encoding for the pap -5 operon DNA sequence, | |
| | which resulted from the ligation pHUR849 linearized with | |
| | Hind III. to a 3.85 Hind III DNA fragment derived from | |
| | pKD849-4 which contains a 300-bp deletion of papH | |

^{a}MCS of SK- is 657-759 bp, and is flanked by T3 and T7 promoters.

Nucleic acid isolation and manipulations

Large scale plasmid DNA isolation was carried out using a QIAGEN Plasmid Kit (QIAGEN, Inc., Chatsworth, Calif.). Small scale plasmid mini-preps used for routine DNA analysis were performed using the alkaline lysis method 6). Each protocol provided DNA of sufficient purity to obtain reproducible co-amplification PCR results. The lysozyme boiling miniprep method (7) was used for the isolation of double-stranded DNA templates for sequencing.

Restriction endonucleases, T4 DNA ligase, Kienow fragment of polymerase I, T4 DNA polymerase, and deoxynucleoside-triphosphates were used according to the conditions recommended by the commercial suppliers (New England BioLabs, Beverly, Mass., and Boehringer Mannheim, Indianapolis, Ind.). After digestion with restriction endonucleases, DNA fragments larger than 1 kb were separated by electrophoresis on 0.7% agarose gels; whereas, separation of smaller DNA fragments was done on 1.5 or 2% agarose gels. Electrophoresis was carried out in TAE buffer (40 mM Tris acetate with 1 mM EDTA [pH 8.0]). DNA fragments were isolated from agarose gels using GENE CLEAN (BIO 101 Inc., La Jolla, Calif.), or QAlquick Gel Extraction (QIAGEN, Inc., Chatsworth, Calif.), according to recommendations of the manufacturer.

Oligonucleotide synthesis

Oligonucleotide primers were synthesized by standard phosphoramidite chemistry on a 345 DNA/RNA synthesizer (Applied Biosystems, Foster City, Calif.). After de-blocking at room temperature for 24 h, the primers were recovered by precipitation at room temperature in 1/10 vol of 3 M NaOAc pH 5.2, 2 Vol 100% ETOH. After centrifugation, the pellets were dried under vacuum and resuspended in 200 μl of distilled water. The amount of nucleic acid was estimated by their absorbance at 260 nm. All samples were adjusted to the same concentration. Table 2 lists the thirteen different DNA primers used for both sequencing and PCR analysis. PCR amplification was used to determine the orientation of sub-clones containing deletions within the papH gene of each construct.

DNA sequencing and sequence analysis

Double-stranded DNA sequencing was performed using the dideoxynucteotide chain-termination method (8), using [α³⁵S]-thio-dATP (1000 Ci mmol⁻¹, Amersham, Arlington Heights, Ill.), and T7 DNA polymerase (Sequenase, U.S. Biochemicals, Cleveland, Ohio). Sequencing reactions were performed in both directions to confirm the analysis. The oligonucleotide primer 200aRE was used for sequence analysis of the final deletion constructs pKD200A-8 and pKD210B-11. The oligonucleotide PapHRE was used for sequence confirmation of the final deletion constructs pKD201B-8 and pKD849-5. Analysis of DNA and protein sequences used programs distributed through the University of Wisconsin Genetics Computer Group. Nucleotide and Amino Acid sequences were aligned with LINEUP and PRETTY programs (9).

TABLE 2
Primers used in this study

| Primers | Oligonucleotide sequence | Description |
|---|---|---|
| T3 | 5′ ATTAACCCTCACTAAAG 3′ | anneals to multiple cloning site of SK- |
| T7 | 5′ AATACGACTCACTATAG 3′ | anneals to multiple cloning site of SK- |
| Reverse | 5′ AACAGCTATGACCATG 3′ | anneals to multiple cloning site of SK- |
| PpHFD | 5′ ATGAGACTGCGATTCTCTGT 3′ | anneals to the TAG translational start |
| | | region of all 4 pap H genes |
| PapHRE | 5′ TCCGTTTCTCACAATTCTGA 3′ | anneals to bp 509-526 of the pap H |
| | | gino of pDAL201B, pap-21 and |
| | | pHUR 849. pap-5 |
| 210bFD | 5′ CCTGAAATACGAGAATATTA 3′ | anneals 93-bp upstream of the TAG |
| | | translational stan region at the pap A |
| | | gene of pHUR849, pap-5 (2) |
| 210bRE | 5′ TAATATTCTCGTATTTCAGG 3′ | the complement oil 210bFD and |
| | | anneals to the earns 93-bp region as |
| | | described for 210bFD |
| FOR210b | 5′ TGGACTGGTATAACAATCGA 3′ | anneals 2.9 kb upstream of the TAC |
| | | transational start region of the pap H |
| | | gene of pDAL210B, pap-21 |
| 200aRE | 5′ TCCGTTTCGCACAATTCTGA 3′ | anneals to bp 511-528 at the pap H |
| | | gene of pDAL210B, pap-H, and pap |
| | | 200a, respectively |
| PapFOR^{a} | 5′ ACTGGATTCATGCAGCATTTCT | anneals to bp 258-270 of the pap A |
| | AGAAA 3′ | gene of pHUR849. pap-5 (2) |
| FORSEQ | 5′ TGGACCTCCTGAGCTA 3′ | anneals to bp 456-474 of the pap A |
| | | g&le of pHUR849. pap-5 (2) |
| PapREV^{b} | 5′ GGGGCAGCCCTGCCGTCCCAA | anneals to bp 122-142 of the papH |
| | AT 3′ | gene of pHUR849. pap-B |
| REVSEO | 5′ AAACACCATGAAACACACA 3′ | anneals to bp 41.61 of the pap H gene |
| | | of pHUR849 |

^{a}contains a single Bam HI restriction site single underlined.
^{b}contains a single Sma I blunt end restriction site double underlined.

DNA amplification

DNA amplification was carried out with 50 ng of plasmid DNA: 0:75 μM of each oligonucleotide in distilled H₂O, supplemented with 1% Triton X-100, 2 mM MgCI₂, 200 μM each dNTP, and 1.25 U Taq DNA Polymerase (Promega, Madison, Wis.), in a final volume of 100 μl. PCRs were performed in a Epicomp DNA Thermal Cycler (Epicomp, San Diego, Calif.). All manipulation were carried out with dedicated DNA-free pipettes using Elkay filter pipet tips (Applied Scientific, San Francisco, Calif.), in a sterile field to minimize the risk of contamination. All reagents were added together except for the Taq DNA Polymerase. The reaction mixture was overlaid with 100 μl of sterile mineral oil and was denatured in the thermal cycler at 95° C. for 2 min. Then, Taq polymerase was added and amplification was earned out over 35 cycles, as follows: a 2 mm denaturation step at 94° C., a 1 min annealing step at 50° C., a 1 min primer extension step at 72° C., and finally, products were extended for 7 mm at 72° C. The reaction mixture was held at room temperature until required. Blank control tubes containing all reagents except the template DNA or primers were also run. The amplified DNA fragments were electrophoresed on 1.5% agarose in TAE butter and visualized by staining with ethidium bromide, and the products were photographed under UV light. PCR was used to establish the correct orientation of the DNA fragments bearing the 237 or 300-bp deletions of the papH gene of each of the four deletion derivatives. The amplification of each construct employed at least one or more different pairs of DNA primers. For each deletion derivative, the DNA primers were as follows: REVERSE and 200aRE for pKD200A-7, REVERSE and PapHRE for pKD201B-7, PapFOR and 200aRE or 210bRE for pKD210B-9, and PapFOR and PapHRE for pKD849-4.

Electron Microscopy

A single colony of each papH mutants, their parent recombinants, and the original wild type strains was isolated from a 18 h 37° C. growth on agar, suspended in 500 ul of saline, and processed for standard negative staining for transmission electron microscopy. Also, the broth culture of each bacterial strain was processed for negative staining for observation in the electron microscope.

Hemagglutination Assay

Binding properties of strains were determined by slide agglutination using human P1 erythrocyctes as described previously (10). Also, the agglutination of purified pili was performed as described by Normark et al (11), using 2-fold serial dilution's starting at 500 μg/ml of protein. A positive reaction was determined macroscopically.

Pili Purification

After 24 h incubation at 37° C. in selective broth medium, bacteria were harvested by centrifugation and pili were purified essentially according to the method of Korhonen et al (12). The purity of pili preparations was analyzed on SDS-PAGE by silver staining.

Immunoreactivity

The purified pili from each papH mutant were assessed for immunoreactivity against polyclonal murine and rabbit antibody reactivity in standard ELISA tests and polyclonal murine and rabbit antibody in Western blotting tests.

Vaccine Efficacy

The efficacy of purified pili from each papH mutant was assessed in the standard experimental BALB/c model of pyelonephritis. Cohorts of 20 female mice that were 14 weeks old were immunized intramuscularly on day 0 and day 14 with 50 ug of purified pili from each papH mutant, as determined by Lowry technique. Each vaccinal administration consisted of 100 ul of pili-incomplete Freund's adjuvant emulsion. Mice were challenged intravesicularly on day 30 by 10⁶ bacteria expressing the homologous pili antigen. Challenge strains included: J96 for KD849-5 vaccine recipients; 3669 for KD2001-8 vaccine recipients; KD201 for KD201-8 vaccine recipients; and KD210B for KD210B-11 vaccine recipients. Protection against renal colonization by the challenge strain was assessed at day 2 after challenge. Positive controls included cohorts of 5 non-vaccinated mice challenged with each strain of bacteria. The pili vaccine conferred protection if the right renal homogenates did not reveal any bacterial growth in >90% of the cohort and none of the renal homogenates in the cohort had more than 5 CFU per gram of tissue. For comparative purposes, all right kidney homogenates from control animals needed to have >100 CFU of the challenge strain per gram of tissue.

### Results

Nucleotide sequences and deduced PapH primary structures

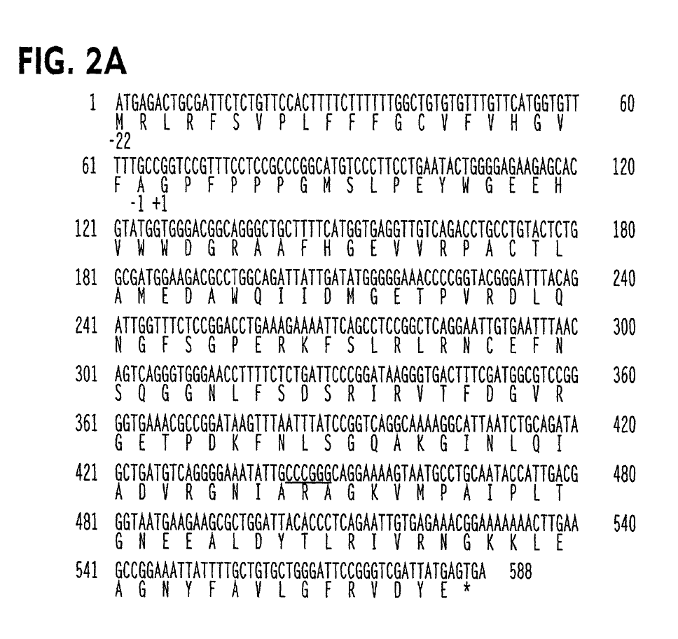

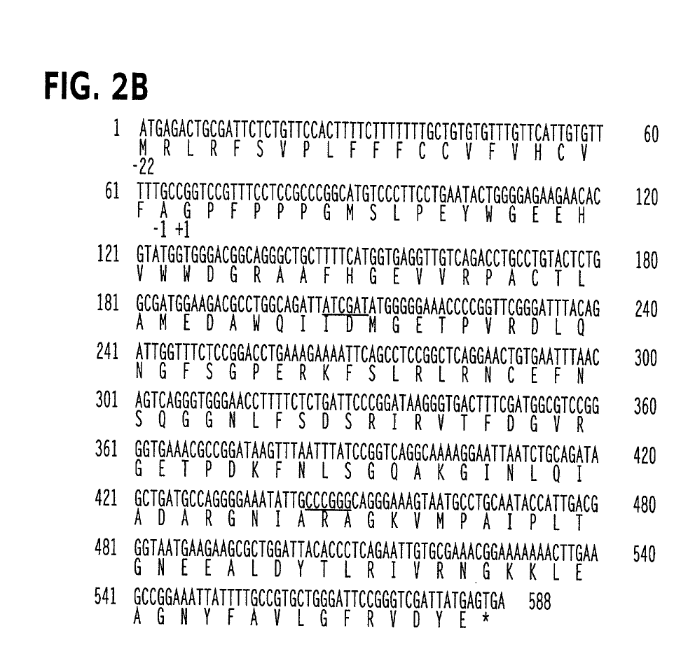

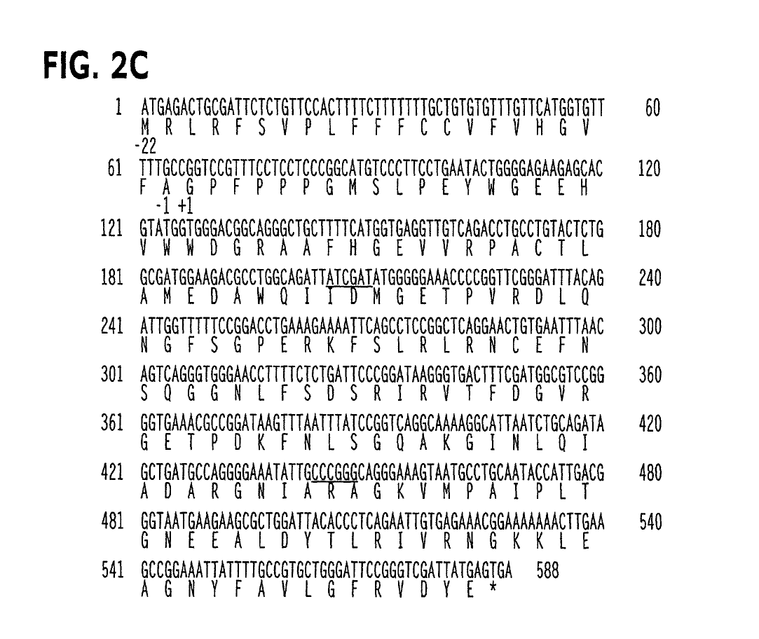

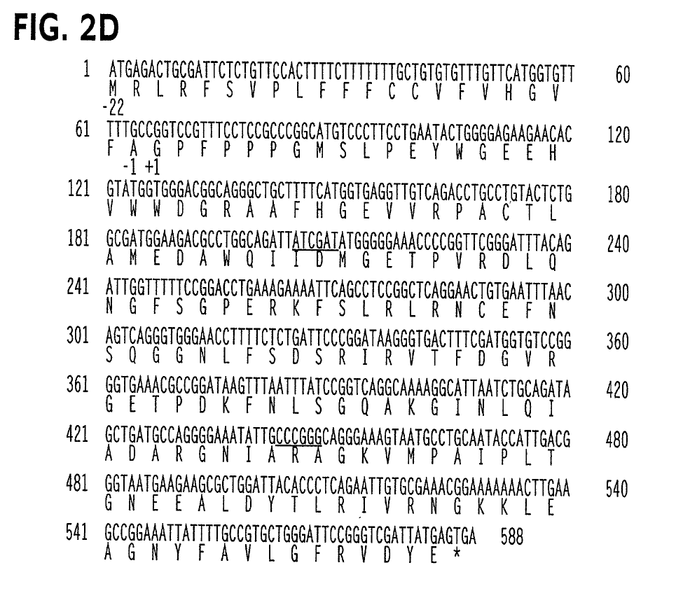

The plasmids pHUR849 (pap-5), pDAL201B (pap-21), pDAL210B (pap-17) and, pDAL200A (pap-200A), in E coli strain HB101 express digalactose-binding of the serotypes F13, F7₁, F7₂ and F9, respectively. The pap gene cluster responsible for regulation and biogenesis of these pili from E. coli strains J96, C1212 and, 3669 is diagrammed in FIG. 1. Sequence analysis of paph genes from pDAL201B (pap-21), pDAL210B (pap-17) and, pDAL200A (pap-200A), was compared to the known nucleotide sequence of papH gene of pHUR849 (pap-5) (3). FIGS. 2 shows a single 588-bp open reading frame with the same polarity as papA (2, 4). Analyses of these papH sequences revealed many typical features of prokaryotic gene organization. All four papH gene sequences contained a potential ribosome-binding sites, ATG initiation codon signal sequence, and a TGA termination codon. A potential initiation codon ATG at position −22, preceded by a sequence corresponding to -AGGGT, which showed homology to ribosome-binding sites, was found 13-bp upstream in all four papH sequences. A protein initiated here and ending at the TGA triplet at position 586 would encode a 195 amino acid polypeptide with a calculated molecular weight of 21.9 kd. The mature PapH protein contains 173 amino acid residues. The NH₂-terminal amino acid sequence of the open reading frame has all the features of a signal peptide sequence. The deduced putative signal sequence for the papH was located 22 codons upstream of their terminal Ala (FIG. 2). These sequences contained a highly hydrophobic region comprising an amino acids stretch of Ser-Val-Pro-Leu-Phe-Phe-Phe. There was a positively charge amino acid residue (Arg) at the position −21. The suggested cleavage sites between Ala −1 and gly +1 conforms to rules of prokaryotic signal cleavage sites and was similar to most other bacterial genes (12). In addition, the final papH deletion derivatives, pKD849-5 (pap-5), pKD201B (pap-21), pKD210B-11 (pap-17) and pKD200A-8 (pap-200A), were also sequenced. In addition, sequencing into the papA and papC genes which flank the papH gene (FIG. 1) of all four papH deletion derivatives was carried out in order to insure that all three genes were in frame. Finally, the codon usage of the paph genes of pDAL201B, pDAL210B and, pDAL200A, and papH gene of pHUR849 were analyzed using a codon frequency computer program (13). The pattern of codon utilization was not significantly different among the genes.

Comparison of papH nucleotide and PapH amino acid sequences

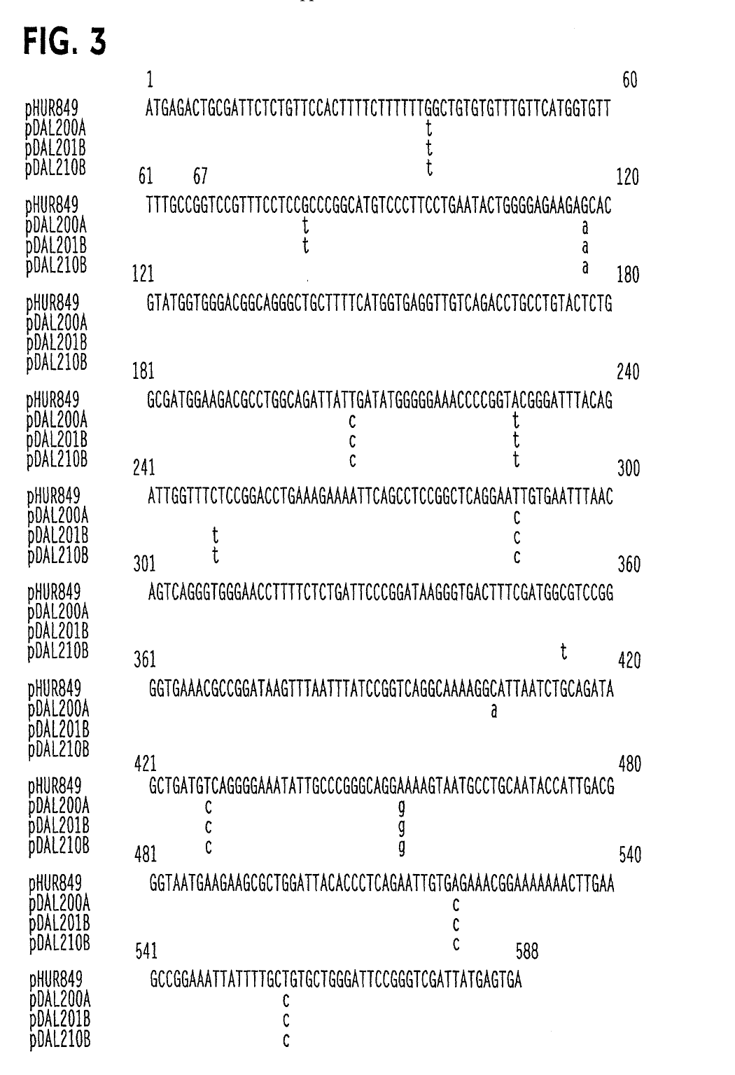

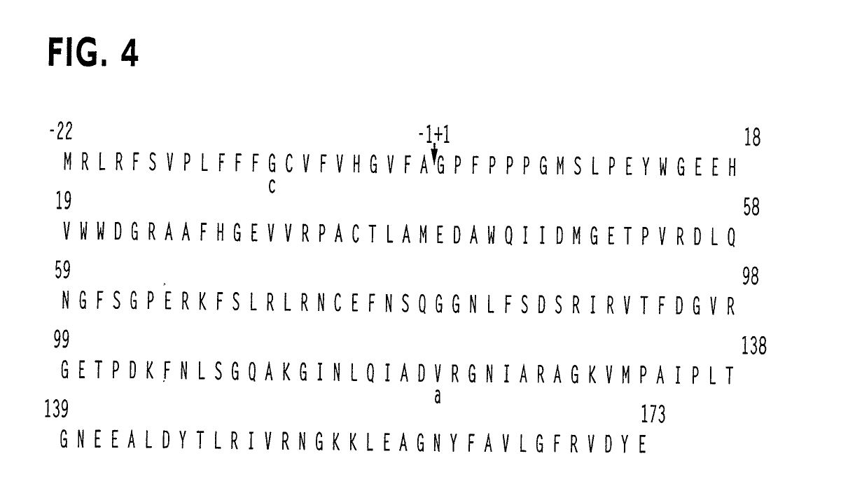

LINEUP and PRETTY computer programs (9) were used to calculate the overall percent homology of the predicted PapH polypeptide at the nucleotide and amino acid level. FIGS. 3 and 4 compare the deduced nucleotide and amino acid sequences of the papH genes of pDAL201B, pDAL210B and, pDAL200A, to the known nucleotide and amino acid sequences of the papH gene of pHUR849. The overall homology among pDAL201B, pDAL210B and, pDAL200A, papH genes was greater than 99% at the nucleotide level and 100% at the amino acid level. Compared to the nucleotide sequence corresponding of papH gene of pHUR849, there was 98% and 99% homology at the nucleotide level and amino acid level, respectively. The amino acid differences among these three papH genes were evaluated and compared to the amino acid sequence of the papH gene for pHUR849 and are shown in FIGS. 4. Comparisons of the substituted amino acid between pDAL201B, pDAL210B, pDAL200A and, pHUR849 papH genes shows that there were only two amino acid changes among these genes, and both these changes were non conserved amino acid substitutions. The first non-conserved substitution is Gly→Cys, at amino acid residue −13 of the putative signal sequence, and the second non-conserved substitution Is Val→Ala at amino acid residue 121 of the mature PapH protein. The deduced mature PapH protein of 173 amino acid residues shares many structural features with known E. coil pilins. PapH contains two cysteine residues 38 amino acids apart in the NH₂- terminal half of the protein, a tyrosine residue as the penultimate amino acid, and shows an overall sequence similarity to other E. Coli pilins, especially from Gly 23 to Gly 50 and in the COOH-terminal region (3). These data show that there is genetic conservation of the papH genes among these strains at both the nucleotide and amino acid level.

PapH deletion mutants

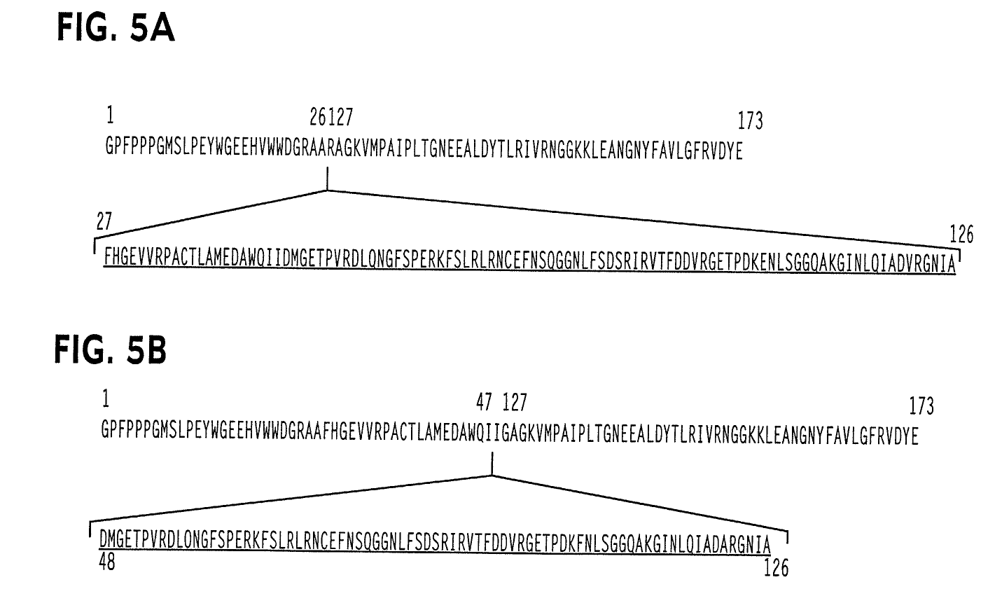

FIG. 5 compares the deduced amino acid sequence of the papH deletion mutants, pKD201B-B, pKD210B11, and pKD200A-8, to the amino acid sequence of the deletion mutant of pKD849-5. The final construct pKD849-5 (pap-5), contains a 300-bp deletion (nucleotides 145-445, FIG. 2), which encodes for 100 amino acids residues (R 27-126). This deletion mutant now contains an open reading frame (ORF) of 219-bp which encodes for a mature fusion protein of 73 amino acids. As shown in FIG. 5, the final constructs pKD201B-8 (pap-21), pKD210B-11 (pap-17) and, pKD200A -8 (pap-200A), all contain a 237-bp deletion (nucleotides 207-445, FIG. 2), which encodes for 79 amino acids residues (R 48-126), respectively. All three mutants, carry ORF's of 282-bp, which encodes for a mature fusion protein of 94 amino acids. The 94 amino acids are identical among these constructs. In addition, pKD201B-8, pKD210B-11 and pKD200A-8 each contain, one non- conserved amino acid substitution at amino acid residue 121 (Val→Ala). Among these strains, this single amino acid substitution within each gene due to a change in the coding sequence of papH gene. This alteration in the coding sequence of the final constructs (Table 1, FIGS. 2 and 5), arose following the self-ligations of the end-filled Cla I to Sma I restrictions sites in the intermediate constructs pKD201B-3, pKD210B-9 and pKD200A-3, respectively.

Electron Microscopy

Each of the negative stained papH mutants viewed under the transmission electron microscope revealed essentially few if any protruding pili-like structures from the cell surface. In contrast, negative stained bacteria from the parent recombinants and their original wild type strains reveal great numbers of pili-like structure protruding from the cell surface under similar condition of preparation. Also, broth cultures from each papH mutant processed for electron microscopy revealed pili free of cellular debris.

Pap H Mutations Affect Cell Association of Pap Pili

E. coli strain HB101 harboring pHUR849, pDAL201B, pDAL210B, and pDAL200a and their papH mutants, pKD849-5, pKD201B-8, pKD210B-11, and PkD200A-8, respectively, were assessed for hemagglutination. A single CFU of each strain grown on agar agglutinated human P1 erythrocytes. Also, purified pili from the recombinant strains with an intact pap operon or a papH mutant agglutinated human P1 erythrocytes. The culture supernatants of papH mutants were sufficient to hemagglutinate P1 erythrocytes; whereas, the parenteral recombinants would only hemaggluttinate erythrocytes when pelleted cells were used. These results suggest that papH mutants secrete functional Gal-Gal pili into culture supernatant prepared from these strains.

Pili Isolation and Pili Characterization from papH Mutants

The pili from each papH mutant purified by a standard method (10) revealed a single band in SDS-PAGE corresponding to the putative PapA moiety of the respective PapA of the parent recombinant and their original wild type strain.

The amount of purified pili from each papH mutant strain obtained from 1 liter 18 h broth culture was estimated to be >10 mg. This was calculated by taking the average of 3 protein determinations of aliquots of known diluted purified pili preparation. A Lowry technique was used to estimate protein concentration.

Each purified pili preparation from papH mutants was bound by homologous murine and rabbit antisera raised against whole pili of the respective parent recombinant and their original wild type strain in ELISA tests. Identical binding patterns and kinetics were observed among the pili preparations from papH mutants, recombinant pili, and wild type pili with these antisera. Also, identical immunoreactivity of the purified pili from papH mutants was demonstrated by Western blots with murine and rabbit antisera elicited against whole pili of the respective parent recombinant and their original wild type.

In addition, cohorts of mice immunized with purified pili from each papH mutant were protected from subsequent renal colonization./infectivity by the challenge strain. None of the control mice were protected from renal colonization/infectivity by the challenge strain.

Summary

We have mutagenized the papH gene of 4 Gal-Gal pilus recombinants, pHUR849 (pap-5), pDAL201B (pap-21), pDAL210B (papl7), and pDAL200A (pap-200A). These recombinants encode for the serotypes F13, F71, F72, and F9 (2) respectively. They are intended to be used in large-scale pili vaccine production. This was accomplished by cloning the papH gene of these recombinant strains to determine the nucleotide and deduced amino acid sequence of these genes. When compared to the previously published nucleotide and amino acid sequence of the papH gene of pHUR849 (3), the recombinant stains pDAL201B, pDAL210B, and pDAL200A were 98% and 99% homologous at the nucleotide level and amino acid level respectively. Based on these sequences, employing PCR and standard recombinant DNA techniques, we were able to create specific deletions within each papH gene. This resulted in 4 recombinant deletion derivatives of pHUR849, pDAL201B, pDAL210B, and pDAL200A, known as pHUR949-5, pDAL201B-8, pDAL210B-11, and pDAL2-A-8. The recombinantpKD849-5, contains a 330-bp deletion that encodes for 100 amino acid residues. This deletion derivative contains an open reading frame of 219-bp that encodes for a mature fusion protein of 73 amino acids. The final constructs pKD201B-8, pKD210B-11, and pKD200A-8 carry 237-bp deletion that encodes for an identical mature fusion protein of 94 amino acids. In addition, we also sequenced into the papA and papC genes that flank the papH gene of all four deletion derivatives to insure that all three genes were in frame for each recombinant. This extended sequence analysis was vital for two reasons. First, the papA , papH, and papC genes are co-regulated at the transcription level. The transcriptional activity of these genes is dependent on the transcriptional activity of papl and papB genes (3,14-17). Second, mutations in both papA and papC completely abolish pilus formation and the expression of the adhesin on the cell surface; whereas, mutations in papC alone do not affect the pilin antigen produced within the cells (18).

Broth culture results using the E. coli bacterial strain HB101 containing these deletion derivatives show that these constructs now release newly synthesized pili fibers into the culture medium. Moreover, these results are consistent with other studies on 30 the regulation and biogenesis of Pap pili (1, 3, 11, 14-20). In studies, carried out by Nomark and co-workers (3, 18), they have shown that in two different mutations in the papH gene in one strain that 50%-70% of the total pilus antigen was found free of the cells in the culture supernatant in the form of a polymerized structure. Our results would further indicate that high amounts of newly synthesized pili fibers are released into the supernatant by these papH mutants. It is also important to note that these pili from the papH mutants have identical PapA pilin molecular weights as their parent recombinant and wild type strain. Furthermore, they are immunological similar to the parent recombinant pili and wild type pili by allowing specific antibody binding to occur in ELISA tests and Western blots. From a vaccine production perspective, these pili can be readily isolated and purified and retain their protective vaccine capacity as demonstrated by their eliciting protection against experimental BALB/c pyelonephritis. Since our data confirm the highly conserved nature of the anchoring gene of Gal-Gal binding piliated bacteria, this strategy of mutagenizing the anchoring gene of the pilus by deletions or other means will be used for other Gal-Gal binding pili belonging to classical F8, F10, F11, F12 serotypes and variant piliated strains (F1C) and uncharacterized pili types in order to collect dissociated pili in broth culture.

Cited References

1. Johnson, Clin. Microbiol. Rev., 4(1990): 80-128;

2. Denich et al., Infect. Immun., 59(1991): 3849-3858;

3. Baga et al., Cell, 49(1987): 241-251;

4. Hull et al., Infect. Immun., 33(1981): 933;

5. Hanahan, Mol. Biol., 166(1983): 557-580;

6. Birmboim et al., J. Nucl. Acid Res., 7(1979): 1513-1523;

7. Holmes et al., Anal. Biochem., 114(1981): 192-197;

8. Sanger et al., Proc. Natl. Acad. Sci. USA, 74(1977): 5463-5467;

9. Lipman et al., Science, 227(1985): 1435-1441;

10. Denich et al., Infect. Immun., 59(1991): 2089-2096;

11. Normark et al., Infect. Immun., 41(1983): 942-949;

12. Korhonen et al., Infect. Immun., 27(1980): 569-575;

13. Devereux, J. Nucl. Acid. Res., 12(1985): 378-395;

14. Norgren et al., EMBO J., 3(1984): 1159-1165;

15. Blyn et al., EMBO J., 9(1990): 4045-4054;

16. Braaten et al., J. Bact., 173(1991): 1789-1800;

17. Braaten et al., Proc. Natl. Acad. Sci. USA, 89(1992): 4250-4254;

18. Norgren et al., J. Mol. Biol., 1(1987): 169-178;

19. Linberg et al., Nature, 328(1987): 84-87; and

20. Baga et al., J. Bact., 157(1984): 330-333.

The invention has been described above with reference to specific examples. Further modifications and variations known to those of ordinary skill based on the description herein are contemplated to be within the invention.

The disclosures of all cited references are expressly incorporated herein to the same extent as if each was individually incorporated by reference.

What is claimed is:

1. An immunogenic composition comprising dissociated pili from a pilus-producing bacteria having at least one mutation that facilitates detachment of the pili from the bacteria relative to a wild type strain and a pharmaceutically acceptable carrier.

2. A vaccine for preventing urinary tract infections comprising an immunogenic composition as claimed in claim 1.

3. A process for producing pili comprising culturing a pilus-producing bacteria having at least one mutation that facilitates detachment of the pili from the bacteria relative to a wild type strain and recovering dissociated pili.

4. A process for producing a vaccine comprising formulating a vaccine comprising pili produced according to claim 3.

5. A method of treating or preventing a urinary tract infection comprising administering to a subject in need thereof a vaccine of claim 2.
